(19) United States
(12) Patent Application Publication
(10) Pub. No.: US 2006/0063250 A1
(43) Pub. Date: Mar. 23, 2006

(54) Use of modified novirhabdoviruses to obtain vaccines

(75) Inventors: Michel Bremont, Choisy-le Roi (FR); Stephane Biacchesi, Villiers-Sur-Marne (FR); Monique Leberre, Montigny-Le-Bretonneux (FR); Maria- Isabel Thoulouze, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: Institut National de la Recherche Agronomique, Paris (FR)

(21) Appl. No.: 10/513,436

(22) PCT Filed: May 7, 2003
(86) PCT No.: PCT/FR03/01413

§ 371 Date: Jun. 27, 2005

(30) Foreign Application Priority Data
May 7, 2002 (FR) 02/05702

(51) Int. Cl. A61K 39/205; C12N 7/00

(52) U.S. Cl. 435/235.1; 424/224.1

(57) ABSTRACT

The invention concerns the use of modified novirhabdoviruses wherein the NV protein gene is inactivated to obtain vaccines for use in particular in fish.

The present invention relates to the use of novirhabdoviruses for producing vaccines.

Novirhabdoviruses are negative-strand RNA viruses of the Rhabdoviridae family.

The Novirhabdovirus genus comprises various pathogenic species for aquatic animals, among which mention will in particular be made of two species that are pathogenic for fish, and mainly for the Salmonidae: the infectious hematopoietic necrosis virus (IHNV) and the viral hemorrhagic septicemia virus (VHSV).

The structure of the novirhabdoviral genome is similar to that of mammalian rhabdoviruses, but differs therefrom by the presence of an additional gene, encoding a non-structural protein, called NV (for “non-virion”) protein, the function of which remains unknown at the current time.

The novirhabdoviral genome comprises six genes, the organization of which can be represented diagrammatically as follows:
3′-N-P-M-G-NV-L-5′

N represents the gene encoding the nucleoprotein associated with the viral RNA, P represents the gene encoding the phosphoprotein associated with the viral polymerase, M represents the gene encoding the matrix protein, G represents the gene encoding the envelope glycoprotein G, NV represents the gene encoding the NV protein, and L represents the gene encoding the RNA-dependent viral RNA polymerase.

IHNV and VHSV cause considerable damage in fish farming, in particular among the young fish. Vaccines capable of conferring protective immunity against these viruses have been proposed. They are based either on the use of killed or inactivated viruses, or on the use of glycoprotein G, which can induce the synthesis of neutralizing antibodies capable of conferring protective immunity. It has thus been proposed to use vaccines based on recombinant glycoprotein G, or naked DNA vaccines carrying the gene encoding glycoprotein G. However, these vaccines must be administered by injection, and it is very difficult in practice to use this method of administration on thousands of young fish.

Previous studies by the team of the inventors (BIACCHESI et al., J. Virol., 74, 11247-11253, 2000) have shown that, when the NV gene of the IHN virus is inactivated and replaced with a reporter gene such as the gene encoding GFP (green fluorescent protein), the recombinant viruses thus produced conserve their infectious capacity and can multiply normally in cell culture.

The inventors have now discovered that, surprisingly, these recombinant IHN viruses lacking the NV protein gene completely lose their pathogenic capacity. They have also noted that, if the NV gene of the VHS virus is inserted in place of the NV gene of origin, the pathogenicity of the virus is re-established; on the other hand, if the VHS virus glycoprotein G gene is inserted in place of the NV gene of origin, non-pathogenic infectious viruses are obtained that are capable of inducing protective immunity against both the IHN virus and the VHS virus. In addition, they have noted that, although the recombinant IHN viruses lacking the NV protein gene conserve their ability to multiply in cell culture, they do not multiply in fish (or are eliminated very rapidly).

This discovery makes it possible to propose the use of novirhabdoviruses in which the NV protein gene is inactivated, for expressing, in vivo, one or more exogenous genes of interest, in a fish.

A subject of the present invention is more particularly the use of novirhabdoviruses in which the NV protein gene is inactivated, for obtaining medicinal products.

The expression “novirhabdoviruses in which the NV gene is inactivated” is intended to mean any novirhabdovirus carrying a mutation which results in the absence of production of the NV protein, or the production of a nonfunctional NV protein, without abolishing the production or the function of the other viral proteins.

The NV protein gene can, for example, be inactivated by deletion of at least one part of said gene or, advantageously, of all of it, or optionally by nonsense mutation, reading frame shift, etc. The inactivation will generally preferably be carried out by deletion of all or of a considerable portion of the gene, which makes it possible to avoid any risk of reversion to the wild-type virus.

In particular, a subject of the present invention is any recombinant novirhabdovirus in which the NV protein gene is inactivated, and which contains in its genome at least one heterologous gene encoding a protein of therapeutic interest.

According to a preferred embodiment of the present invention, said protein of therapeutic interest is chosen from vaccine antigens of organisms, and in particular of viruses, that are pathogenic for fish, and in particular from capsid or envelope glycoproteins of viruses that are pathogenic for fish. By way of nonlimiting examples, mention will be made of glycoprotein E2 and glycoprotein El of the salmonid sleeping disease virus (VILLOING et al., J. Virol. 74, 173-183, 2000, and Dis. Aquat. Org., 40, 19-27, 2000) or glycoprotein G of a rhabdovirus, in particular glycoprotein G of a novirhabdovirus of a species other than that from which the recombinant novirhabdovirus in accordance with the invention is derived.

According to a preferred embodiment of the present invention, said heterologous gene is inserted at the position of the inactivated NV protein gene.

Advantageously, a recombinant novirhabdovirus in accordance with the invention is chosen from:

an IHN virus in which the NV protein gene is inactivated, and replaced with the glycoprotein G gene of a VHS virus;

a VHS virus in which the NV protein gene is inactivated, and replaced with the glycoprotein G gene of an IHN virus.

Recombinant novirhabdoviruses in accordance with the invention can be obtained by means of methods known in themselves, in particular by means of the method described in U.S. Pat. No. 6,033,886 for preparing rhabdoviruses, which consists in cotransfecting a host cell expressing RNA polymerase with the complementary DNA (cDNA) of the viral genome, and DNA molecules encoding the N, P and L viral proteins.

The cDNA of the genome of a recombinant novirhabdovirus in accordance with the invention is obtained from the cDNA of the corresponding wild-type virus by introduction of one or more mutations that inactivate the NV gene, and insertion of the heterologous gene of interest. These modifications can be introduced by means of conventional genetic engineering techniques.

The present invention also encompasses the cDNA of the genome of a recombinant novirhabdovirus in accordance with the invention, and also any recombinant vector comprising said cDNA.

A subject of the present invention is also the medicinal products comprising a novirhabdovirus in which the NV gene is inactivated, and in particular the vaccines comprising a recombinant novirhabdovirus in accordance with the invention.

The vaccines in accordance with the invention can be used in fish, and in particular salmonids, such as farmed trout and salmon.

The use of recombinant novirhabdoviruses in accordance with the invention, which simultaneously express glycoprotein G of the novirhabdovirus of origin and at least one vaccine antigen derived from another pathogenic organism, make it possible to obtain multivalent vaccines capable of conferring protection against at least two pathogens. For example, a recombinant novirhabdovirus in accordance with the invention which expresses, besides glycoprotein G of the novirhabdovirus from which it is derived, glycoprotein G of a novirhabdovirus of a different species makes it possible to confer protection against the two species of novirhabdovirus in question.

In addition, unlike the vaccines proposed in the prior art, which can only be administered by injection, the vaccines in accordance with the invention have the advantage of also being able to be administered by balneation, i.e. by simple addition of the vaccine to the water in the breeding tank containing the animals to be immunized. This method of administration is therefore much simpler to carry out than the method by injection. In addition, it makes it possible to solve the problems posed by the immunization of the young fish, which are the most sensitive to the diseases induced by novirhabdoviruses, but also to the stress associated with immunization by injection.

Furthermore, the lack of multiplication in the fish of the recombinant novirhabdoviruses in accordance with the invention allows them to be used without any risk of dissemination in the environment or of contamination of wild-type species.

The present invention will be understood more thoroughly from the further description which follows, which refers to nonlimiting examples of construction and of use of recombinant novirhabdoviruses in accordance with the invention.

### EXAMPLE 1

### Construction of Recombinant Novirhabdoviruses by Replacement of the NV Gene With a Heterologous Gene

Construction of the recombinant cDNAs:

The constructions were carried out using the plasmid pIHNV described by BIACCHESI et al. (J. Virol. 2000 74(23):11247-53). This plasmid contains the complete cDNA of the IHN virus genome, cloned downstream of the T7 phage RNA polymerase promoter and upstream of a ribozyme sequence of the hepatitis δ virus and of the T7 phage RNA polymerase transcription terminator, in the vector pBlueScript SK (Stratagene).

A recombinant IHN virus in which the NV gene was excised and replaced with the GFP (Green Fluorescent Protein) reporter gene was constructed as described by BIACCHESI et al. (publication mentioned above).

Diagrammatically:

An SpeI restriction site and a SmaI restriction site were introduced by site-directed mutagenesis (Quickchange kit, Stratagene) into an EagI-PstI fragment of the cDNA of the IHN virus genome on either side of the NV open reading frame. This open reading frame was excised from this fragment by SpeI-SmaI digestion, and replaced with the GFP reporter gene. The modified EagI-PstI fragment was introduced into the plasmid pIHNV in place of the corresponding region thereof.

The plasmid obtained is called pINHV-ΔNV-GFP.

Recombinant IHN viruses in which the NV gene was replaced with the NV gene or G gene of VHS were constructed as follows:

The NV and G genes were obtained from the 07-71 strain of VHS (SCHUETZE H. et al. Virus Genes., 19(1), 59-65, 1999; GenBank NC 000855), using the following primers: NV gene:

| | |
|---|---|
| 3′ primer: | |
| SmaI | |
| CCCGGGTCAGGAGGTGAGCCCAGAGCC | (SEQ ID NO: 1) |
| 5′ primer: | |
| SpeI | |
| ACTAGTATGGCGACCCAACCCGGGCTCAGC | (SEQ ID NO: 2) |
| G gene: | |
| 3′ primer: | |
| SmaI | |
| CCCGGGTCAGACCGTCTGACTTCTGGAGAACT | (SEQ ID NO: 3) |
| 5′ primer: | |
| SpeI | |
| ACTAGTATGGAATGGAACACTTTTTTCTTGG | (SEQ ID NO: 4) |

Carp epithelial cells (EPC: epithelioma papulosum cyprinid) are infected with the VHS virus at a multiplicity of infection of 1. Twenty-four hours post-infection, the cells are lyzed and the total RNAs are extracted (RNAgents Total RNA Isolation System kit, PROMEGA). Approximately 1 μg of RNAs are denatured for 10 min at ambient temperature in the presence of 40 mM of hydroxymethyl mercury. The denatured RNA is then placed in a reaction mixture containing 80 mM of β-mercaptoethanol, 10 mM of dithiothreitol, 1 mM of dNTP, 40 U RNasin (GIBCO-BRL), 25 pmol of the VHSV G 3′ primer or of the VHSV NV 3′ primer, and 200 U of SUPERSCRIPT II, in buffer IX (GIBCO-BRL). The mixture is kept at 42° C. for 1 hour.

The cDNAs obtained are amplified by PCR with the VHSV G 5′ primer or the VHSV NV 5′ primer, in the presence of TAQ polymerase (GIBCO BRL). The PCR products thus obtained are purified and subcloned into a TA-cloning plasmid(pGEM-T Vector System, PROMEGA).

After sequencing, the VHSV G or NV DNA fragments are excised by SpeI and SmaI digestion.

One or other of these fragments is inserted, in place of the NV gene, into the EagI-PstI fragment of the cDNA of the IHN virus genome, and the EagI-PstI fragment thus modified is introduced into the plasmid pIHNV as indicated above.

The plasmids obtained are called pINHV-ΔNV-NVSHV and pINHV-ΔNV-GSHV, respectively.

Production of the Recombinant Viruses

Three expression plasmids comprising respectively the genes encoding the nucleoprotein N, the phosphoprotein P, and the RNA-dependent RNA polymerase L of IHN were constructed, as described by BIACCHESI et al. (publication mentioned above) . These constructs are respectively called pT7-N, pT7-P and pT7-L.

The plasmid pIHNV, the plasmid pINHV-ΔNV-NVSHV or the plasmid pINHV-ΔNV-GSHV, and the 3 plasmids, pT7-N, pT7-P and pT7-L, at respective doses of 1 μg, 0.5 μg, 0.2 μg and 0.2 μg) are introduced, by transfection in the presence of lipofectamine (GIBCO-BRL), into EPC cells infected beforehand with a recombinant vaccinia virus expressing the T7 phage RNA polymerase (vTF7-3, FUERST et al. Proc. Natl. Acad. Sci. USA, 92, 4477-4481, 1986).

After transfection, the cells are incubated for 5 hours at 37° C. and then washed with MEM culture medium (without serum) and incubated for 7 days at 14° C. in MEM culture medium containing 2% of fetal calf serum. The cells and the supernatant are frozen/thawed, and clarified by centrifugation for 10 minutes at 10 000 rpm. The supernatant is used at a 1/10 dilution to infect a layer of EPC cells. The viruses are produced in the supernatant 3-4 days post-infection.

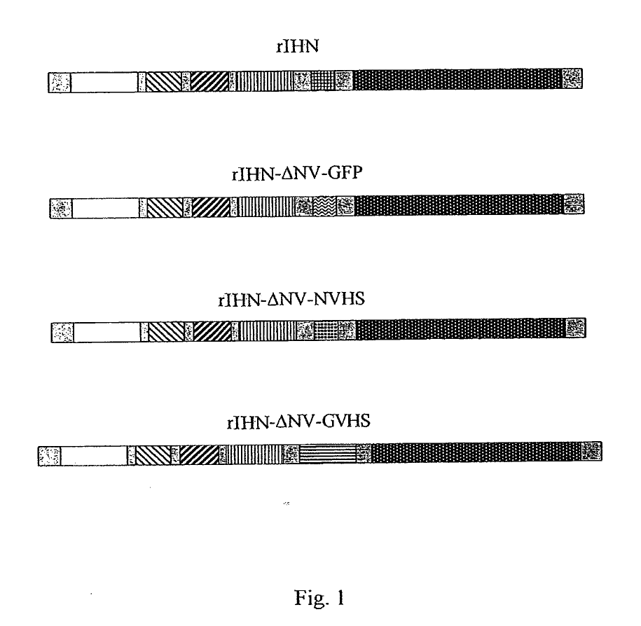

The structure of the viruses obtained, having the entire genome of the wild-type virus (rIHN), or resulting from the deletion of the NV gene and its replacement with the gene encoding GFP (rIHN-ΔNV-GFP), the NV gene of VHSV (rIHN-ΔNV-NVVHS), or the G gene of VHSV (rIHN-ΔNV-GVHS), is shown diagrammatically in FIG. 1.

Legend of FIG. 1:

| | |
|---|---|
| [symbol] | Intergenic regions |
| □ | N gene |
| ▪ | P gene |
| □ | M gene |
| ▪ | G gene IRN |
| ▪ | G gene VHS |
| ▪ | NV gene IHN |
| □ | NV gene VHS |
| ▪ | GFP gene |

Viral stocks of each of the viruses produced were constituted by successive passages in (EPC) cell culture of the supernatant taken 7 days after transfection (supernatant PO). The cells are infected at a multiplicity of infection (m.o.i.) of 1. After 3 passages, the supernatants are removed at various times post-infection, and titrated by limiting dilution in order to establish a growth curve.

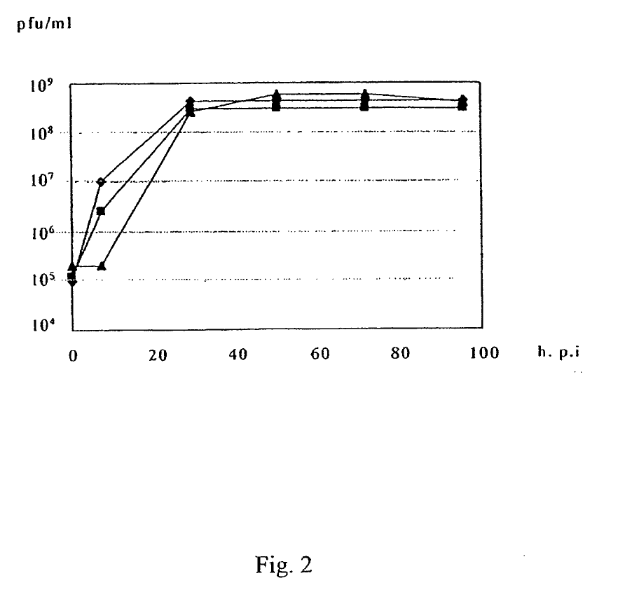

The growth curves for the wild-type VHS and IHN viruses, and for the recombinant virus IHNV-ΔNV-GVHSV are represented in FIG. 2. These curves show that the recombinant virus IHNV-ΔNV-GVHSV multiplies in cell culture as well as the wild-type IHN or VHS viruses.

Legend of FIG. 2:

♦: wild-type VHS virus;

▪: wild-type IHN virus;

▴: recombinant virus IHNV-ΔNV-GVHSV.

### EXAMPLE 2

### Pathogenicity and Vaccine Properties of the Recombinant Novirhabdoviruses

Pathogenicity

The pathogenicity of the various novirhabdoviruses obtained as described in Example 1 above was evaluated by experimental infections in rainbow trout (Oncorhyncus mykiss).

Infection by Injection:

Batches of 25 young trout/tank (average weight 2 g) were infected by injection of the various viruses tested at a rate of 10⁶ pfu/fish. The viruses used are as follows:

Wild-type IHN virus, French strain 32/87 (wt 32/87)

Wild-type IHN virus produced as described in Example 1 (rIHN);

Virus IHN-ΔNV-GFP (rIHN-ΔNV-GFP) ;

Virus IHN-ΔNV-NVVHS (rIHN-ΔNV-NVVHS)

Virus IHN-ΔNV-GVHS (rIHN-ΔNV-GVHS)

The mortality is monitored over a period of 4 weeks.

The results observed in 2 independent experiments are summarized in Table 1 below.

TABLE I

| VIRUS | Mortality★ |
|---|---|
| wt 32/87 | ++ |
| rIHN | ++ |
| rIHN-ΔNV-GVHS | − |
| rIHN-ΔNV-GFP | − |
| rIHN-ΔNV-NWHS | ++ |

★Cumulative mortalities between 7 and 15 days post-infection (++) 90-100%; (−) <10%.

Infection by Balneation

Young trout fish were infected by balneation, according to the following protocol:

The young fish are placed in breeding tanks in a small volume of water (3 liters of water per 100 to 150 young fish). The virus to be tested is added to the water of the tank in a proportion of 10⁴ to 5×10⁴ PFU/ml (PFU =plaque forming unit) . After incubation for 3 hours, the tanks are filled and the water circulation is re-established.

The viruses used are as follows:

wtIHNV: wild-type IHN virus, French strain 32/87

wtVHSV: wild-type VHS virus, strain 07-71;

wtSVCV: spring viremia of carp virus (vesiculovirus incapable of multiplying in trout), Fijan strain (FIJAN et al., Veterinarski archiv (Zagreb), 41, 125138, 1971; complete sequence: HOFFMAN et al., GenBank AJ318079) ;

rIHNV: wild-type IHN virus produced as described in Example 1;

G-VHS: recombinant IHN virus carrying the G gene of the VHS virus in place of the G gene of IHN;

G-SVCV: recombinant IHN virus carrying the G gene of the SVCV virus in place of the G gene of IHN;

G-IHN-VHS: recombinant IHN virus carrying the G gene of the VHS virus in place of the NV gene of IHN;

ΔNV-GFP: recombinant IHN virus carrying the GFP gene in place of the NV gene of IHN;

NVVHS: recombinant IHN virus carrying the NV gene of the VHS virus in place of the NV gene of IHN.

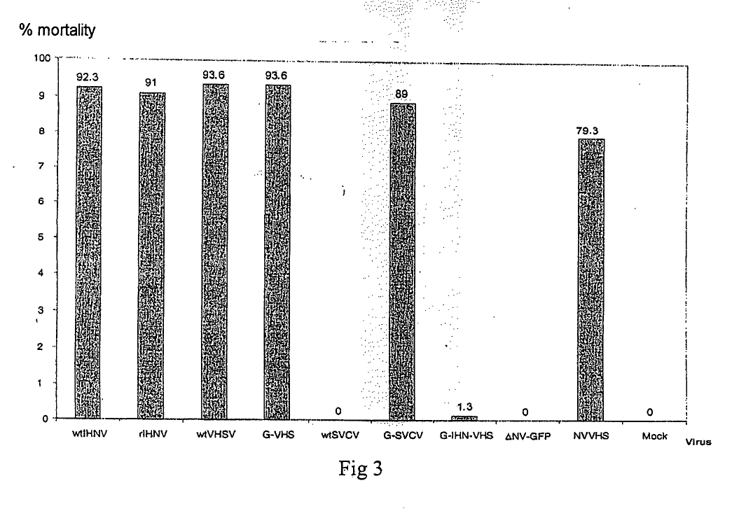

The results are illustrated in FIG. 3.

Legend to FIG. 3

X-axis: virus used; mock: uninfected young fish

Y-axis: cumulative mortality at 28 days post-infection (as percentage of the initial number of young fish).

These results show that the pathogenicity of the viruses is linked to the presence of the NV gene; all the viruses containing this gene result in a high mortality of the young fish; on the other hand, all the viruses in which the NV gene was inactivated lost their pathogenic capacity.

Vaccine Properties

Fish (young trout fish) immunized by balneation, under the same conditions as in the pathogenicity trials, with the virus rIHN-ΔNV-GVHS or the virus rIHN-ΔNV-GFP were subjected, 1 month later, to an infection formed by balneation with the wild-type IHNV and VHSV viruses, at a rate of 10⁴ to 5×10⁴ PFU/ml.

No significant mortality was observed in the case of the fish immunized with the virus rIHN-ΔNV-GVHS. The fish immunized with the virus rIHN-ΔNV-GFP are also protected against the IHNV virus. On the other hand, they die between 7 and 15 days after infection with the VHSV virus.

These results show that the immunization with the rIHN-ΔNV-GVHS virus effectively protects both against the wild-type IHNV virus and against the wild-type VHSV virus.

1. A recombinant novirhabdovirus in which wherein the NV protein gene is inactivated.

2. A recombinant novirhabdovirus wherein the NV protein gene is inactivated, and wherein the recombinant novirhabdovirus contains in its genome at least one heterologous gene encoding a vaccine antigen of an organism that is pathogenic for fish.

3. The recombinant novirhabdovirus as claimed in claim 2, wherein said heterologous gene encodes a capsid or envelope glycoprotein of a virus that is pathogenic for fish.

4. The recombinant novirhabdovirus as claimed in claim 2, wherein said heterologous gene is inserted at the position of the NV gene.

5. The recombinant novirhabdovirus as claimed in claim 4, wherein said novirhabdovirus is chosen from
an infectious hematopoietic necrosis (IHN) virus in which the NV protein gene is inactivated, and replaced with the glycoprotein G gene of a viral hemorrhagic septicemia (VHS) virus;
a VHS virus in which the NV protein gene is inactivated, and replaced with the glycoprotein G gene of an IHN virus.

6. A complementary DNA molecule (cDNA) of the genome of a recombinant novirhabdovirus as defined in claim 1.

7. A recombinant vector comprising a cDNA molecule as claimed in claim 6.

8. A vaccine comprising a recombinant novirhabdovirus as claimed in claim 1.

9. A vaccine comprising a recombinant novirhabdovirus as defined in claim 2.

10. The vaccine as claimed in claim 8, for preventing novirhabdovirus infections in fish.

11. A method for producing a medicinal product comprising adding a recombinant novirhabdovirus as claimed in claim 1 to a medicament.

12. The vaccine as defined in claim 9 for preventing novirhabdovirus infections in fish.

13. A complementary DNA molecule (cDNA) of the genome of a recombinant novirhabdovirus as defined in claim 2.

14. A recombinant vector comprising a cDNA molecule as claimed in claim 13.

15. A method for producing a medicinal product comprising adding a recombinant novirhabdovirus as claimed in claim 2 to a medicament.


SEQUENCE LISTING (4 sequences)

SEQ ID NO: 1 - DNA, 27 bp, Artificial Sequence
  synthetic oligonucleotide
cccgggtcaggaggtgagcccagagcc

SEQ ID NO: 2 - DNA, 30 bp, Artificial Sequence
  synthetic oligonucleotide
actagtatggcgacccaacccgggctcagc

SEQ ID NO: 3 - DNA, 32 bp, Artificial Sequence
  synthetic oligonucleotide
cccgggtcagaccgtctgacttctggagaact

SEQ ID NO: 4 - DNA, 31 bp, Artificial Sequence
  synthetic oligonucleotide
actagtatggaatggaacacttttttcttgg